(19) United States
(12) Patent Application Publication
(10) Pub. No.: US 2021/0073889 A1
(43) Pub. Date: Mar. 11, 2021

(54) METHOD AND SYSTEM FOR ASSISTING A USER IN REAL-TIME FOR SELECTING PRODUCTS

(71) Applicant: TOSHIBA TEC KABUSHIKI KAISHA, Tokyo (JP)

(72) Inventors: Kathiresan SELVARAJ, Bangalore (IN); Rahul Gupta, Bangalore (IN)

(73) Assignee: TOSHIBA TEC KABUSHIKI KAISHA, Tokyo (JP)

(21) Appl. No.: 16/676,242

(22) Filed: Nov. 6, 2019

(30) Foreign Application Priority Data
Sep. 11, 2019 (IN) 201941036542

(51) Int. Cl. G06Q 30/06; G16H 20/60; G06T 19/00

(52) U.S. Cl. CPC G06Q 30/0627; G06Q 30/0631; G06T 19/006; G16H 20/60; G06Q 30/0641

(57) ABSTRACT

The present disclosure relates to a method and an assisting system for assisting a user in real-time for selecting products. The method and system involve receiving information of a set of nutrients associated with a product selected by the user. Thereafter, health data and a health goal associated with the user are received from a user profile database. Information of the set of nutrients is then analyzed based on a health effect of pre-stored nutrients similar to the set of nutrients. Based on the analysis, one of a first expected impact or a second expected impact of the set of nutrients is identified based on the health data. The selected product is then detected as suitable for the user upon identifying the first expected impact of the set of nutrients on the health data of user.

### CROSS-REFERENCE TO RELATED APPLICATION

This application is based upon and claims the benefit of priority from Indian Patent Application No. 201941036542, filed on Sep. 11, 2019, the entire contents of which are incorporated herein by reference.

### TECHNICAL FIELD

The present disclosure generally relates to data processing for product selection. More particularly, but not exclusively, the present disclosure relates to a method and a system for assisting a user in real time for selecting products.

### BACKGROUND

At present, using a mobile device before or during shopping has become part of a decision making process for customers. Customers may use the mobile device for various reasons such as comparing prices, viewing product ratings, viewing product reviews, viewing information on product suitability, and the like.

Customers may also use retail applications provided by retail stores. Using these retail applications, the retail stores may engage the customers by providing information such as advertisements of one or more products in the store, new products added to the store, discounts or offers available on one or more products, and the like. However, providing a retail application which is personalized for the users and understands needs of health conscious customers remains a challenge.

The information disclosed in this background section is only for enhancement of understanding of the general background of the present disclosure and should not be taken as an acknowledgement or any form of suggestion that this information forms the prior art already known to a person skilled in the art.

### SUMMARY

Disclosed herein is a method of assisting a user in real-time for selecting products. The method comprises receiving, by an assisting system in a device associated with a user, information of a set of nutrients associated with a product selected by the user. The method also comprises retrieving health data associated with the user comprising at least one of: one or more health conditions associated with the user, one or more medical conditions associated with the user, one or more medications associated with the user, or one or more health goals associated with the user. The health data is retrieved from a user profile database associated with the assisting system. The method further comprises analyzing the information of the set of nutrients based on a health effect of pre-stored nutrients similar to the set of nutrients. Based on the analysis, the method comprises identifying one of a positive impact or a negative impact of the set of nutrients on the health data associated with the user. Thereafter, the method comprises detecting the selected product as suitable for the user upon identifying the positive impact of the set of nutrients on the health data associated with the user.

Further, the present disclosure discloses an assisting system for assisting a user in real-time for selecting products. The assisting system comprises a processor and a memory communicatively coupled to the processor. The memory stores the processor-executable instructions, which, on execution, causes the processor to receive information of the set of nutrients associated with a product selected by the user. The instructions also causes the processor to retrieve health data associated with the user comprising at least one of one or more health conditions associated with the user, one or more medical conditions associated with the user, one or more medications associated with the user and a health goal associated with the user from a user profile database associated with the assisting system. Thereafter, the instructions causes the processor to analyze the information of the set of nutrients based on a health effect of pre-stored nutrients similar to the set of nutrients. Once the information is analyzed, the instructions causes the processor to identify one of a positive impact or a negative impact of the set of nutrients on the health data associated with the user based on the analysis. Further, the instructions causes the processor to detect the selected product as suitable for the user upon identifying the positive impact of the set of nutrients on the health data associated with the user.

Furthermore, the present disclosure comprises a non-transitory computer readable medium including instructions stored thereon that when processed by at least one processor causes an assisting system for assisting a user in real-time for selecting products by receiving information of the set of nutrients associated with a product selected by the user. The selected product along with the information is displayed in the device using AR technique. Further, the instructions cause the processor to retrieve health data associated with the user comprising at least one of one or more health conditions associated with the user, one or more medical conditions associated with the user, one or more medications associated with the user and a health goal associated with the user from a user profile database associated with the assisting system. Furthermore, the instructions cause the processor to analyze the information of the set of nutrients based on a health effect of pre-stored nutrients similar to the set of nutrients. In addition, the instructions cause the processor to identify one of a positive impact or a negative impact of the set of nutrients on the health data associated with the user based on the analysis. Finally, the instructions cause the processor to detect the selected product as suitable for the user upon identifying the positive impact of the set of nutrients on the health data associated with the user.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of the present disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1a illustrates an exemplary environment for assisting a user in real-time for selecting products in accordance with some embodiments of the present disclosure;

FIG. 1b shows nutrient comparison between the selected product and the suggested product in accordance with some embodiments of the present disclosure;

FIG. 2a shows a block diagram of an assisting system in accordance with some embodiments of the present disclosure;

FIG. 2b and FIG. 2c show exemplary notifications provided in a user device in accordance with some embodiments of the present disclosure;

FIG. 3 shows a flowchart illustrating a process of assisting a user in real-time for selecting products in accordance with some embodiments of the present disclosure; and

FIG. 4 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially implemented in at least one computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the present disclosure is susceptible to various modifications and alternative forms, specific embodiment thereof has been shown by way of example in the drawings and will be described in detail below. It should be understood, however that it is not intended to limit the present disclosure to the specific forms disclosed, but on the contrary, the present disclosure is to cover all modifications, equivalents, and alternative falling within the scope of the present disclosure.

The terms “comprises,” “comprising,” “includes,” “including,” or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure relates to method and system (assisting system) for assisting a user in real-time for selecting a product. At first, a user may select a product and the system may receive information of a set of nutrients associated with the product selected by the user. The user may select the product by scanning the product using an image capturing unit (e.g., image recorder) or a barcode scanner configured in a device associated with the user. As an example, the device may include, but is not limited to, a mobile phone, camera, a tablet, and any other computing device capable of capturing an image and transmitting information associated with the captured image. The system may be configured in the device for assisting the user in real-time in selecting the product. Upon scanning, the system may receive information of (i.e., relating to) the set of nutrients associated with a product. As an example, the information of the set of nutrients may include information comprising a fat level, an energy level, a carbohydrate level, and a protein level in the selected product. In an embodiment, the system may be associated with a user profile database to retrieve health data associated with the user. The health data may comprise at least one of one or more health conditions associated with the user, one or more medical conditions associated with the user, one or more medications associated with the user, or a health goal associated with the user. As an example, the one or more health conditions may be one or more disease types, the one or more medical conditions may be allergic reactions associated with the user for one or more food products and food drug interaction, and the one or more medications may be information associated with medicines being taken by the user.

In an embodiment, the system may analyze the information of the set of nutrients based on a health effect of pre-stored nutrients similar to the set of nutrients. The information associated with the pre-stored nutrients may be stored in a product database associated with the system. The pre-stored nutrients may be associated with one or more products. The product database may also comprise information associated with one or more general health conditions, one or more products comprising pre-stored nutrients which may potentially positively impact the one or more general conditions and one or more products comprising pre-stored nutrients which may potentially negatively impact the one or more general conditions. The system may identify one or more general health conditions from the product database matching with the one or more health conditions associated with the user and also identify pre-stored nutrients from the product database relating to the set of nutrients of the selected product, e.g., pre-stored nutrients that are matching with or similar to the set of nutrients of the selected product. Thereafter, the system may identify an expected positive impact of the set of nutrients on the health data when the identified pre-stored nutrients positively impact the matched one or more general health conditions. The system may identify an expected negative impact of the set of nutrients on the health data when the identified pre-stored nutrients negatively impact the matched one or more general health conditions. If the positive impact on the health data is identified, then the system may detect the selected product as suitable for the user and provide a notification. However, if the negative impact on the health data is identified, the system may detect the selected product as unsuitable and provide a notification to the user. In this manner, the present disclosure assists the user in real-time for selecting a product which is suitable based on a health condition and a health goal associated with the user.

In the following detailed description of the embodiments of the present disclosure, reference is made to the accompanying drawings that form a part hereof, and in which are shown by way of illustration of embodiments in which the present disclosure may be practiced. These embodiments are described in sufficient detail to enable those skilled in the art to practice the present disclosure, and it is to be understood that other embodiments may be utilized and that changes may be made without departing from the scope of the present disclosure. The following description is, therefore, not to be taken in a limiting sense.

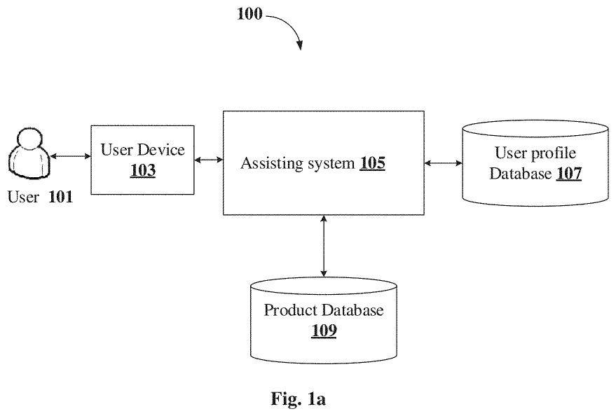

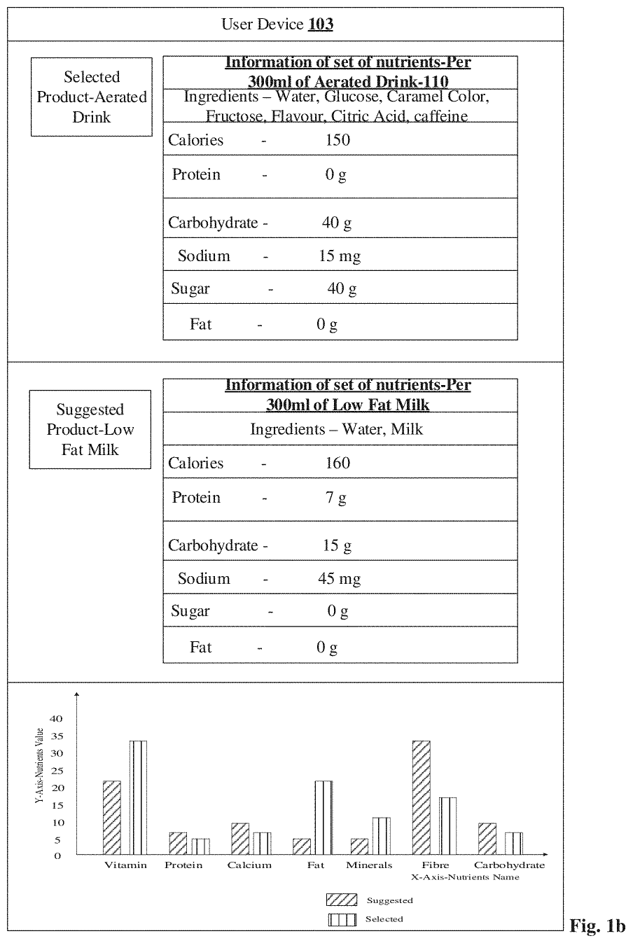

FIG. 1a illustrates an exemplary environment for assisting a user in real-time for selecting a product in accordance with some embodiments of the present disclosure.

The environment 100 may comprise a user 101, a user device 103, an assisting system 105, a product database 109, and a user profile database 107. As an example, the user 101 may be at a retail store to buy one or more products. To this effect, the present disclosure provides a system 105 [also referred as assisting system] which assists the user 101 in real-time for selecting the product suitable for the user 101 based on a health condition and a health goal associated with the user 101. In an embodiment, the system 105 may be provided as an application in and/or on the user device 103. In another embodiment, the system 105 may be associated with the user device 103. For example, the system 105 may be located in a server that is remote relative to the user device 103 and in communication with the user device 103 through a communication network [not shown in FIG. 1a]. The user device 103 may include, but is not limited to, a mobile phone, a tablet, and any other computing device capable of capturing an image and transmitting data associated with the captured image.

As an example, the user 101 may scan the product using an image capturing unit or a bar code scanner in the user device 103. The scanned product may be displayed in the user device 103 along with information 110 of the set of nutrients using Augmented Reality (AR) techniques. The product may be packaged food products or perishable food products. As an example, the product may include, but is not limited to, grains, white rice, oil, snack items, fruits, vegetables, meat, cold drinks, pastry, cereals, wheat flour, and any other consumable product which has effect on health associated with the user 101. Upon scanning, the assisting system 105 may receive information 110 of the set of nutrients associated with the product selected by the user 101. As an example, the user 101 may select a 1 litre “aerated drink” bottle. The selected product may be displayed in the user device 103 along with the information 110 of the set of nutrients [shown in Table 1 below] using AR technique as shown in FIG. 1b.

TABLE 1
Information of set of nutrients-Per 300 ml
Ingredients - Water, Glucose, Caramel Color,
Fructose, Flavour, Citric Acid, caffeine

| Calories | 150 | |
|---|---|---|
| Protein | 0 | g |
| Carbohydrate | 40 | g |
| Sodium | 15 | mg |
| Sugar | 40 | g |
| Fat | 0 | g |

As an example, the information 110 may include, ingredients of the product and nutrient value such as protein value, fat value, carbohydrate value, sugar value, sodium value, and the like.

In order to detect whether the selected product is suitable for the user 101 or not, the system 105 may require information 110 that includes health data and a health goal associated with the user 101. Therefore, the system 105 may retrieve the health data and a health goal associated with the user 101 from the user profile database 107. The user profile database 107 may also include details associated with the user 101 such as username, age, user identification number, and the like. The health data may comprise at least one of one or more health conditions associated with the user 101, one or more medical conditions, and one or more medications. The one or more health conditions may be one or more disease types. The one or more medical conditions may be one or more allergic reactions associated with the user 101 for one or more food products and food drug interaction. The one or more medications may be information of medicines being consumed by the user 101. The health goal associated with the user 101 may be, for example, “weight loss.” An exemplary user profile database 107 may be as shown in below Table 2.

TABLE 2

| | |
|---|---|
| Username | ABC |
| User Age | 40 |
| User ID | 1170 |
| One or more health conditions | High Cholesterol |
| One or more medical conditions | None |
| One or more medications | Statins |
| Health goal | Weight loss |

Upon retrieving the health data and the health goal associated with the user 101, the system 105 may analyze the information 110 of the set of nutrients of the selected product based on a health effect of pre-stored nutrients to the set of nutrients. The analysis may be performed in order to identify one of a positive impact or a negative impact of the set of nutrients on the health data. The pre-stored nutrients may be associated with one or more general products. The information of the one or more general products are stored in the product database 109. The product database 109 may also include information of one or more general health conditions, pre-stored nutrients, a level of each of the pre-stored nutrients positively impacting the one or more general health conditions, one or more first products comprising the pre-stored nutrients, a level of each of the pre-stored nutrients negatively impacting the one or more general health conditions, one or more second products comprising the pre-stored nutrients negatively impacting the one or more general health conditions, pre-stored nutrients suitable and unsuitable for one or more medications, and pre-stored nutrients affecting one or more medical conditions and health goals. The exemplary product database 109 may be as shown in Table 3, below.

TABLE 3

| | | | Pre-stored nutrients | | Pre-stored nutrients | | | |
|---|---|---|---|---|---|---|---|---|
| | | | positively impacting | | negatively impacting | | | |
| | | | the general health | | the general health | | | Prestored nutrients |
| | | | conditions and the | | conditions and the | Pre-stored nutrients | Prestored nutrients | affecting medical |
| General Health | | One or more | level of the pre- | One or more | level of the pre- | suitable for one | unsuitable for one | conditions and |
| Conditions | | first products | stored nutrients | second products | stored nutrients | or more medications | or more medications | health goals |
| 1. | Thyroid | Fish, Dairy | Iodine, Zinc | Sweet Potatoes, Broccoli, | Goitrogens | Iodine | Calcium for | Unsaturated fats |
| | | Products,Egg | | peanuts, cabbage, spinach | | | levothroxine | for weight loss |
| | | | | Recommended Level - Less | | | | |
| | | | | than 1% of daily calories. | | | | |
| 2. | High | Fish(FattyFish - Sardine, | Unsaturated Fats | Red meat (Beef, Pork, Lamb), | Saturated Fats | Fish(Fatty Fish - Sardine, | Calcium, magnesium | Unsaturated fats |
| | Cholesterol | Salmon, Tuna, Trout), plants, | | whole fat/reduced-fat dairy | | Salmon, Tuna, Trout), plants, | in Grape Juice | for weight loss |
| | | tree nuts(walnuts, badam, | | products, Poultry with | | tree nuts(walnuts, badam, | affect Statins | |
| | | hazel nuts, pecans, cashews), | | skin, saturated vegetable | | hazel nuts, pecans, cashews), | | |
| | | legumes, seeds (chia, | | oils (coconut oil, palm oil), | | legumes, seeds (chia, | | |
| | | flaxseeds), beans, and | | Lard, Shortening, Sausage, | | flaxseeds), beans, and | | |
| | | vegetable oils(olive oil, | | bacon, organ meats | | vegetable oils(olive oil, | | |
| | | canola oil), fat-free/ | | Recommended Level - Less | | canola oil), fat-free/ | | |
| | | low-fat milk/yogurts, | | than 5% of daily calories. | | low-fat milk/yogurts, | | |
| | | dark chocolate, cocoa, | | Refined/Simple Carbs - | Simple | dark chocolate, cocoa, | | |
| | | soy foods. | | refined grains, white flour, | Carbohydrate | soy foods. | | |
| | | | | refined Atta, sweetened | | | | |
| | | | | drinks, white rice, and | | | | |
| | | | | cereals with added sugar | | | | |
| | | | | Recommended Level - Less | | | | |
| | | | | than 2% of daily calories. | | | | |
| | | | | Hydrogenated oil (solidified | Trans Fat | | | |
| | | | | vegetable oils), Fried food, | | | | |
| | | | | baked goods, packaged foods, | | | | |
| | | | | Microwaveable popcorns, | | | | |
| | | | | packaged cookies, cakes, | | | | |
| | | | | donuts, pastries, chips, | | | | |
| | | | | crackers, buttered popcorn, | | | | |
| | | | | frosting. | | | | |
| | | | | Recommended Level-less | | | | |
| | | | | than 1% of daily calories. | | | | |

The system 105 may analyze the information 110 of the set of nutrients based on a health effect of pre-stored nutrients similar to the set of nutrients. To perform this analysis, the system 105 may first compare a health condition associated with the user 101 with one or more general health conditions stored in the product database 109. As an example, the health condition associated with the user 101 may be “high cholesterol.” The system 105 may identify the health condition as matching with the one or more general health conditions in the product database 109. Once the health condition is matched, the system 105 may retrieve information corresponding to the matched health condition from the product database 109. The matched health condition “high cholesterol” may be associated with one or more first products comprising the pre-stored nutrients, a level of each of the pre-stored nutrients which positively impacts the health condition, one or more second products comprising the pre-stored nutrients, and a level of each of the pre-stored nutrients which negatively impacts the health condition associated with the user 101. The matched health condition may also be associated with the pre-stored nutrients which are suitable and unsuitable for the health condition, one or more medical conditions, and a health goal associated with the user 101. As shown in Table 3, for the matched health condition “high cholesterol,” the pre-stored nutrients such as “simple carbohydrates” may not be suitable. However, the set of nutrients in the selected product “aerated drink” may comprise “simple carbohydrates” which may affect the health condition associated with the user 101 and also health goal associated with the user 101. Hence, the system 105 may detect the selected product as unsuitable and provide a notification to the user 101. The notification may be provided in the form of text, audio, video, images, animations, and/or emojis. Since the selected product is identified as unsuitable, sad emojis, stickers, or animations may be displayed to demotivate the user 101 using AR technique. For example, when the user selects, the “aerated drink” bottle, the selected product along with the information 110 of the set of nutrients and other information such as suitability and unsuitability of the selected product based on a health condition and a health goal associated with the user 101 is displayed in the user device 103.

Further, since the selected product is detected as unsuitable, the system 105 may suggest one or more alternative products which may be suitable for the user 101. In order to suggest an alternative product, the system 105 may look at the product database 109 to identify one or more products which are categorized as positively impacting the health condition associated with the user 101. As shown in Table 3, for the matched health condition “high cholesterol,” the pre-stored nutrients such as “unsaturated fats” may be suitable as it positively impacts the health condition and a health goal associated with the user 101. The one or more food products which comprises the nutrients “unsaturated fats” may be “dark chocolate,” “walnut,” cashew,” low-fat milk, and the like. Therefore, the system 105 may suggest these products to the user 101 instead of the product “aerated drink.” These products may be suggested along with happy emojis, stickers, or animations to encourage or motivate the user 101 to select these products. As an example, the system 105 may suggest the product “low-fat milk.” The suggested product along with information of the set of nutrients associated with the suggested product “low-fat milk” may be as shown in FIG. 1b.

Further, in an embodiment, the system 105 may identify nutrient level of the set of products selected by the user 101. The system 105 may compare the nutrient level with a daily nutrient level which is suggested to the user 101. If the nutrient level increases the daily nutrient level, the system 105 may provide an indication to the user 101. FIG. 1b also shows an exemplary comparison chart among the set of nutrients in the selected product and the set of nutrients in the set of products selected by the user.

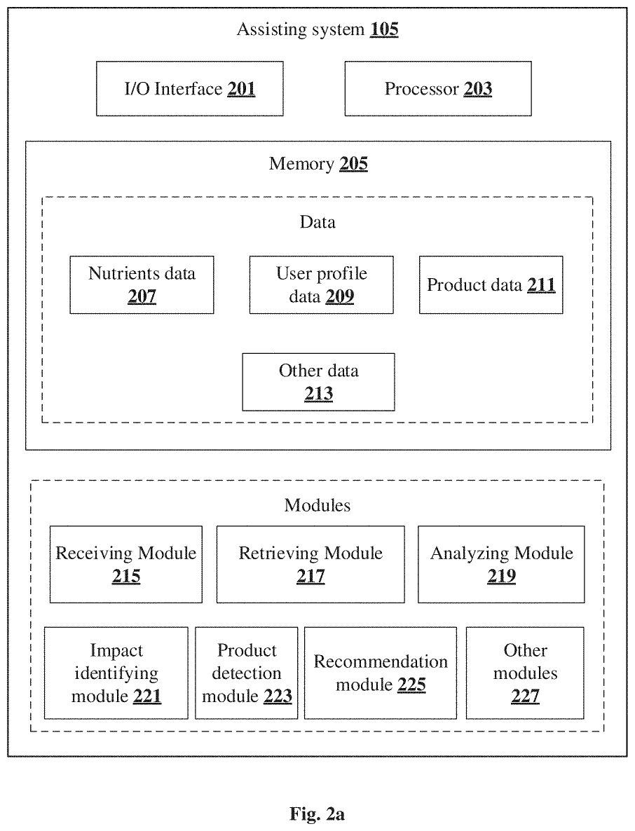

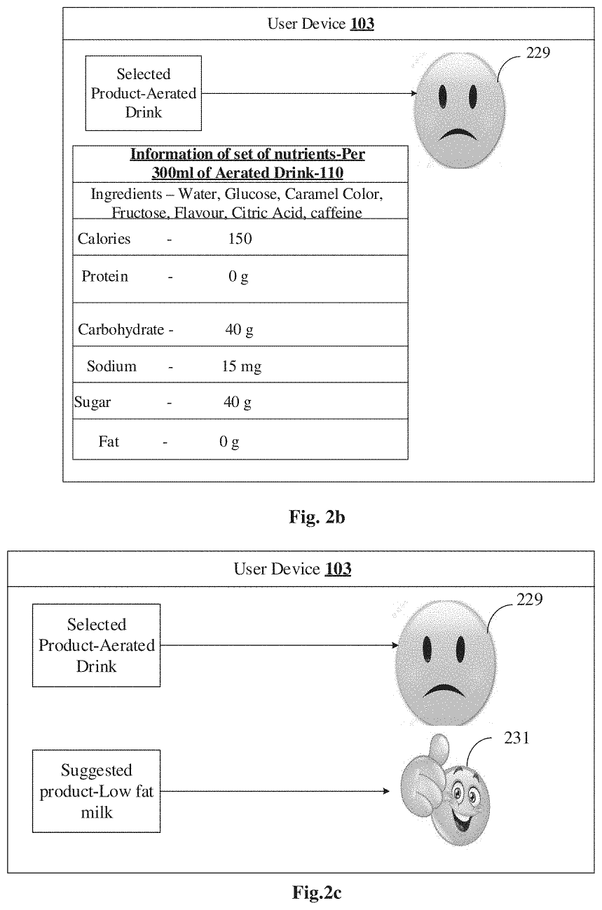

FIG. 2a shows a block diagram of an assisting system in accordance with some embodiments of the present disclosure.

As shown in FIG. 2a, the assisting system 105 may comprise an Input/Output (I/O) interface 201, a processor 203, and a memory 205. The I/O interface 201 may be configured to receive information from the image capturing device or the barcode scanner or any other device capable of capturing an image or reading data from a bar code and transmitting data or the captured image to a computing device for processing the data or the captured image. The information may be associated with a set of nutrients of a product selected by a user 101. The I/O interface 201 may also be configured to provide a notification about whether the selected product is suitable or unsuitable for the user 101 based on a health condition and a health goal associated with the user 101. The processor 203 may be configured to perform one or more functions of the assisting system 105. In some implementations, the assisting system 105 may include data and modules for performing various operations in accordance with embodiments of the present disclosure. In an embodiment, the data may be stored within the memory 205 and may include, but are not limited to, nutrients data 207, user profile data 209, product data 211, and other data 213.

In some embodiments, the data may be stored within the memory 205 in the form of various data structures. Additionally, the data may be organized using data models, such as relational or hierarchical data models. The other data 213 may store data, including temporary data and temporary files, generated by the modules for performing various functions of the assisting system 105.

In an embodiment, one or more modules may process the data of the assisting system 105. In one implementation, the one or more modules may be communicatively coupled to the processor 203 for performing one or more functions of the assisting system 105. The modules may include, but are not limited to, a receiving module 215, a retrieving module 217, an analyzing module 219, an impact identifying module 221, a product detection module 223, a recommendation module 225, and other modules 227.

As used herein, the term module refers to an Application Specific Integrated Circuit (ASIC), an electronic circuit, a processor 203 (shared, dedicated, or group), and a memory 205 that execute one or more software or firmware programs, a combinational logic circuit, and/or other suitable components that provide the described functionality. In an embodiment, the other modules 227 may be used to perform various miscellaneous functionalities of the assisting system 105. It will be appreciated that such modules may be represented as a single module or a combination of different modules. Furthermore, a person of ordinary skill in the art will appreciate that in an implementation, the one or more modules may be stored in the memory 205, without limiting the scope of the present disclosure. The said modules when configured with the functionality defined in the present disclosure will result in a novel hardware.

In an embodiment, the receiving module 215 may be configured to receive information 110 of the set of nutrients associated with a product selected by the user 101. As an example, the user 101 may select a product and scan the selected product using an image capturing device or a barcode scanner configured in a user device 103 associated with the user 101. Once the product is scanned, the receiving module 215 may receive the information 110 of the set of nutrients associated with the selected product. The information 110 of the set of nutrients may include, but is not limited to, protein level, sugar level, carbohydrate level, fat level, and the like. The information 110 of the set of nutrients may be stored as nutrient data 207.

In an embodiment, the retrieving module 217 may be configured to retrieve health data associated with the user 101 and a health goal associated with the user 101 from the user profile database 107. The health data and the health goal associated with the user along with other details associated with the user such as a username associated with the user, an age associated with the user, and an identification number associated with the user may be stored as user profile data 209. The user profile database 107 may be associated with the assisting system 105. As an example, the health data may comprise at least one of one or more health conditions associated with the user 101, one or more medical conditions associated with the user 101, or one or more medications associated with the user 101. The one or more health conditions may be one or more disease types such as “high cholesterol,” “thyroid,” “diabetes,” and the like. The one or more medical conditions may be one or more allergic reactions or food drug interactions. The one or more medications may be one or more medicines being consumed by the user 101 for one or more disease types. As an example, the user 101 may be consuming statins for “high cholesterol” and “grape juice” may interact with statins and hence must be avoided. This information may be present as food drug interactions.

In an embodiment, the analyzing module 219 may be configured to analyze the information 110 of the set of nutrients based on a health effect of pre-stored nutrients similar to the set of nutrients. The product database 109 may store information associated with one or more general health conditions such as “thyroid” and “high cholesterol” which is retrieved by the analysing module 219 and stored as product data 211. As an example, only these general health conditions such as “thyroid” and “high cholesterol” are present in the product database 109. However, the product database 109 may also comprise information of other general health conditions as well. Each of the one or more general health conditions are associated with one or more first products and one or more second products. The one or more first products may comprise pre-stored nutrients and a level of the pre-stored nutrients positively impacting the one or more general health conditions. As an example, the general health condition may be “high cholesterol.” The pre-stored nutrients positively impacting the health condition “high cholesterol” may be “unsaturated fats.” The one or more first products comprising the pre-stored nutrients “unsaturated fats” may be “dark chocolates,” “vegetable oil,” “cashew nuts,” and the like. The one or more second products may comprise pre-stored nutrients and level of the pre-stored nutrients negatively impacting the one or more general health conditions. The pre-stored nutrients negatively impacting the health condition “high cholesterol” may be, as an example, “saturated fats.” The one or more second products comprising the pre-stored nutrients “saturated fats” may be “red meat” and “organ meat.”

The system 105 may identify the general health condition matching with the health condition associated with the user 101. As an example, the matched health condition may be “high cholesterol.” The system 105 may then compare the set of nutrients with the pre-stored nutrients associated with the matched general health condition. As an example, the user 101 may have selected the product “red meat” which comprises the nutrient “saturated fat.” In an embodiment, the impact identifying module 221 may compare the nutrient information with the pre-stored nutrient of the matched general health condition. The nutrient “saturated fat” may be categorized as the nutrient negatively impacting the matched general health condition in the product database 109. Therefore, the product detection module 223 may detect the selected product “red meat” as unsuitable for the user 101 and provide a notification to the user 101 indicating unsuitability of the selected product. The notification may be provided in the form of text, video, audio, images, and/or emojis.

In an embodiment, the recommendation module 225 may be configured to recommend one or more products suitable for the user 101 based on a health condition and a health goal associated with the user 101. Since the health condition associated with the user 101 is “high cholesterol,” the recommendation module 225 may retrieve information associated with the pre-stored nutrients which are categorized as positively impacting the health condition associated with the user 101. As an example, the pre-stored nutrients “unsaturated fat” may be categorized as the pre-stored nutrient which positively impacts the health condition associated with the user 101. The one or more first products which includes these pre-stored nutrients may be dark chocolate, walnut, cashew, fish, and the like. Therefore, the recommendation module 225 may suggest these products to the user 101.

Exemplary Scenario

As an example, the user 101 “ABC” selects a product, which is 1 Litre “aerated drink bottle at a retail store. The user 101 scans the product using a bar code scanner in the user device 103. Once the product is scanned, the system 105 receives information 110 of the set of nutrients associated with the selected product. The set of nutrients contained in the selected product is as shown in Table 1. From the user profile database 107, the system 105 retrieves health data and a health goal associated with the user 101 as shown in Table 2 which indicates that the user 101 is diagnosed with “high cholesterol” and the health goal associated with the user 101 is to “reduce weight.” The system 105 compares the health condition associated with the user 101 with one or more general health conditions, wherein information of the one or more general health conditions is stored in the product database 109. The system identifies the matched general health condition from the product database 109. The matched general health condition “high cholesterol” is associated with other information such as pre-stored nutrients, a level of pre-stored nutrients positively impacting the health condition associated with the user 101, one or more first products comprising the pre-stored nutrients positively impacting the health condition associated with the user 101, a level of pre-stored nutrients negatively impacting the health condition associated with the user 101, one or more second products comprising the pre-stored nutrients negatively impacting the health condition associated with the user 101, pre-stored nutrients suitable or suitable for one or more medications, pre-stored nutrients affecting the one or more medical conditions, and a health goal associated with the user 101.

As shown in Table 3, the pre-stored nutrient positively impacting the health condition associated with the user 101 is “unsaturated fat” and the one or more first products comprising this nutrient are “Fish (Fatty Fish—Sardine, Salmon, Tuna, Trout), plants, tree nuts (walnuts, badam, hazel nuts, pecans, cashews), legumes, seeds (chia, flaxseeds), beans, vegetable oils (olive oil, canola oil), fat-free/low-fat milk/yogurts, dark chocolate, cocoa, and the like soy foods.

Similarly, the pre-stored nutrient negatively impacting the health condition associated with the user 101 is “saturated fat,” “trans-fat,” “refined/simple carbohydrate,” and the like. The one or more second products comprising the nutrient “saturated fat” are red meat (beef, pork, lamb), whole fat/reduced-fat dairy products, poultry with skin, saturated vegetable oils (coconut oil, palm oil), lard, sausage, bacon, organ meats, and the like. The recommended level of intake of these one or more second products should be less than 5% of daily calorie intake associated with the user 101. The one or more second products comprising the nutrient “trans-fat” are hydrogenated oil (solidified vegetable oils), fried food, baked goods, packaged foods, microwaveable popcorns, packaged cookies, cakes, donuts, pastries, chips, crackers, buttered popcorn, frosting, and the like. The recommended level of intake of these one or more second products should be less than 1% of daily calorie intake associated with the user 101. The one or more second products comprising the nutrient “refined/simple carbohydrate” are refined grains, white flour, refined atta, sweetened drinks, white rice, cereals with added sugar,

and the like. The recommended level of intake of these one or more second products should be less than 2% of daily calorie intake associated with the user 101.

The pre-stored nutrients unsuitable for one or more medications may be “calcium” and “magnesium” and the one or more products comprising these pre-stored nutrients may be “grape juice.” The pre-stored nutrients such as “unsaturated fat” may affect health goal associated with the user 101.

The system compares the set of nutrients associated with the selected product “aerated drink” with the pre-stored nutrients in the product database 109. The set of nutrients associated with the selected product are, carbohydrate-40 gm, sodium-15 gm, and sugar 40-gm. The nutrients “carbohydrate” in the selected product match with the pre-stored nutrients “simple carbohydrate.” These pre-stored nutrients are categorized as the nutrients negatively impacting the health condition “high cholesterol” in the product database 109. Hence, the selected product is detected as unsuitable for the user 101. Therefore, the system 105 may provide a notification on the user device 103 indicating unsuitability of the selected product for the user 101. The notification may be provided in the form of audio, video, and/or text. As an example, one or more emojis may also be used to provide the notification. As shown in FIG. 2b, a sad emoji 229 may be displayed in the user device 103 for the selected product “aerated drink” along with the information of the set of nutrients in the selected product “aerated drink.”

Further, since the selected product is detected as unsuitable for the user 101, the system 105 recommends one or more products which are suitable for the user 101 based on a health condition and a health goal associated with the user 101. Since the health condition associated with the user 101 is “high cholesterol,” the nutrients positively affecting the health condition associated with the user 101 is “unsaturated fat” and the one or more first products comprising the nutrient “unsaturated fat” are fish (fatty fish such as sardine, salmon, tuna, trout), plants, tree nuts (walnuts, badam, hazel nuts, pecans, cashews), legumes, seeds (chia, flaxseeds), beans, and vegetable oils (olive oil, canola oil), fat-free/low-fat milk/yogurts, dark chocolate, cocoa, and soy foods. Therefore, the system 105 suggests/recommends the user 101 to select “low-fat milk” instead of “aerated drink.” As shown in FIG. 2c, a happy emoji 231 may be displayed in the user device 103 for the suggested product “low-fat milk.”

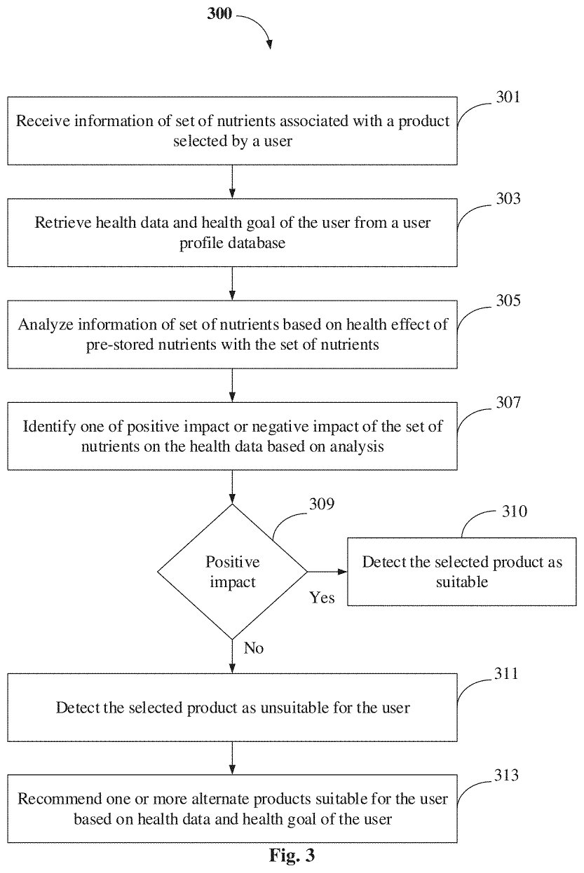

FIG. 3 shows a flowchart illustrating a process of assisting a user in real-time for selecting a product in accordance with some embodiments of the present disclosure.

As illustrated in FIG. 3, the method 300 comprises one or more blocks for assisting a user 101 in selecting products in real-time using an assisting system. The method 300 may be described in the general context of computer executable instructions. Generally, computer executable instructions can include routines, programs, objects, components, data structures, procedures, modules, and functions, which perform particular functions or implement particular abstract data types.

The order in which the method 300 is described is not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

At block 301, the method may comprise receiving information of the set of nutrients associated with a product selected by the user 101. As an example, the user 101 may select a product and scan the selected product using an image capturing device or a barcode scanner configured in a user device 103 associated with the user 101. Once the product is scanned, an assisting system 105 configured in the device may receive the information 110 of the set of nutrients associated with the selected product. The information 110 of the set of nutrients may be as shown in Table 1 for the selected product “aerated drink.”

At block 303, the method may comprise retrieving, by the assisting system, health data and a health goal associated with the user 101 from a user profile database 107. The user profile database 107 may be associated with the assisting system. The health data may comprise at least one of one or more health conditions associated with the user 101, one or more medical conditions associated with the user 101, and one or more medications associated with the user 101. The one or more health conditions may be one or more disease types, the one or more medical conditions may be one or more allergic reactions associated with the user 101 for one or more food products, and the one or more medications may be information of medicines being consumed by the user 101. The health goal associated with the user 101 may be, for example, weight loss.

At block 305, the method may comprise analyzing the information 110 of the set of nutrients based on a health effect of pre-stored nutrients similar to the set of nutrients. To perform this, the system 105 may first compare health condition associated with the user 101 with one or more general health conditions stored in the product database 109. Based on the matched general health condition, the system 105 may identify whether the set of nutrients have either a positive impact or a negative impact on the health condition associated with the user 101.

At block 307, the method may comprise identifying whether the set of nutrients of the selected product positively impact or negatively impact health data associated with the user. The identification may be based on analysis of health effect of pre-stored nutrients similar to the set of nutrients. The identification is performed at block 309. If the set of nutrients are identified to positively impact the health data associated with the user, then the method proceeds to block 310, wherein the selected product is detected as suitable to the user. If the set of nutrients are identified to negatively impact the health data associated with the user then the method proceeds to block 311, wherein the selected product is detected as unsuitable for the user. Thereafter at block 313, the method may comprise recommending one or more alternate products which may be suitable for the health data and a health goal associated with the user.

Further, the system 105 may provide a notification to the user 101 when the selected product is detected as one of suitable or unsuitable for the user 101. The notification may be in the form of text, audio, video, and/or emojis. The system 105 may also suggest/recommend suitable product for the user 101 based on a health condition and a health goal associated with the user 101.

### Computer System

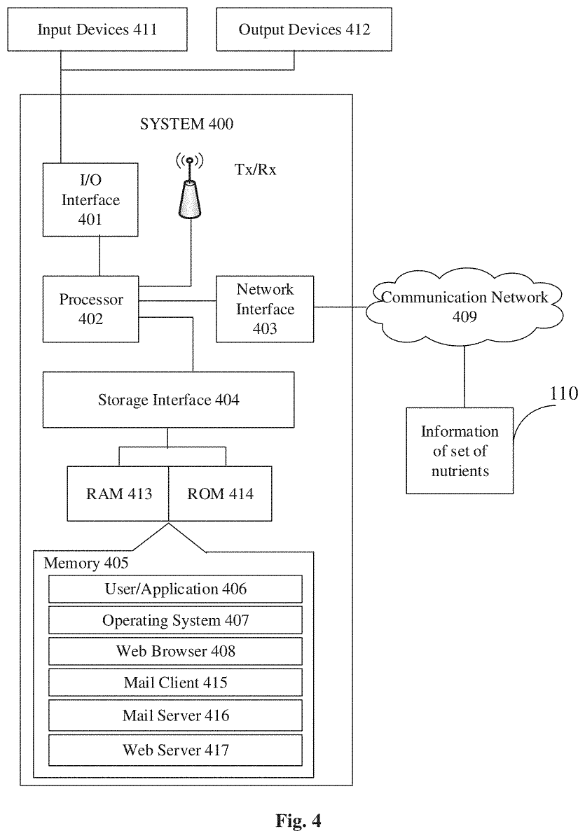

FIG. 4 illustrates a block diagram of an exemplary computer system 400 for implementing embodiments consistent with the present disclosure. In an embodiment, the computer system 400 may be the assisting system 105, which is used for assisting a user 101 in real-time for selecting products. The computer system 400 may include a central processing unit (“CPU” or “processor”) 402. The processor 402 may comprise at least one data processor for executing program components for executing user or system-generated business processes. The processor 402 may include specialized processing units such as integrated system (bus) controllers, memory management control units, floating point units, graphics processing units, digital signal processing units, and the like.

The processor 402 may be disposed in communication with one or more I/O devices (411 and 412) via I/O interface 401. The I/O interface 401 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), and the like. Using the I/O interface 401, the computer system 400 may communicate with one or more I/O devices 411 and 412. In some implementations, the I/O interface 401 may be used to receive information of the set of nutrients 110.

In some embodiments, the processor 402 may be disposed in communication with a communication network 409 via a network interface 403. The network interface 403 may communicate with the communication network 409. The network interface 403 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, and the like.

The communication network 409 can be implemented as one of the several types of networks, such as intranet or Local Area Network (LAN) and such within the organization. The communication network 409 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 409 may include a variety of network devices, including routers, bridges, servers, computing devices, storage devices, and the like.

In some embodiments, the processor 402 may be disposed in communication with a memory 405 (e.g., RAM 413, ROM 414, and the like. as shown in FIG. 4) via a storage interface 404. The storage interface 404 may connect to memory 405 including, without limitation, memory drives, removable disc drives, and the like, employing connection protocols such as Serial Advanced Technology Attachment (SATA), Integrated Drive Electronics (IDE), IEEE-1394, Universal Serial Bus (USB), fiber channel, Small Computer Systems Interface (SCSI), and the like. The memory drives may further include a drum, magnetic disc drive, magneto-optical drive, optical drive, Redundant Array of Independent Discs (RAID), solid-state memory devices, solid-state drives, and the like.

The memory 405 may store a collection of program or database components, including, without limitation, user/application 406, an operating system 407, a web browser 408, mail client 415, mail server 416, web server 417, and the like. In some embodiments, computer system 400 may store user/application data 406, such as the data, variables, records, etc., as described in the present disclosure. Such databases may be implemented as fault-tolerant, relational, scalable, secure databases such as Oracle® or Sybase®.

The operating system 407 may facilitate resource management and operation of the computer system 400. Examples of operating systems include, without limitation, APPLE MACINTOSH® OS X, UNIX®, UNIX-like system distributions (e.g., BERKELEY SOFTWARE DISTRIBUTION™ (BSD), FREEBSD™, NETBSD™, OPENBSD™, etc.), LINUX DISTRIBUTIONS™ (e.g., RED HAT™, UBUNTU™, KUBUNTU™, etc.), IBM™ OS/2, MICROSOFT™ WINDOWS™ (e.g., XP™, VISTA™/7/8, 10 etc.), APPLE® IOS™, GOOGLE® ANDROID™, BLACKBERRY® OS, or the like. A user interface may facilitate display, execution, interaction, manipulation, or operation of program components through textual or graphical facilities. For example, user interfaces may provide computer interaction interface elements on a display system operatively connected to the computer system 500, such as cursors, icons, check boxes, menus, windows, widgets, etc. Graphical User Interfaces (GUIs) may be employed, including, without limitation, APPLE MACINTOSH® operating systems, IBM™ OS/2, MICROSOFT™ WINDOWS' (XP™, VISTA™/7/8, 10 etc.), Unix® X-Windows, web interface libraries (e.g., AJAX′, DHTML™, ADOBE® FLASH′, JAVASCRIPT™, JAVA′, etc.), or the like. Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

### ADVANTAGES OF PRESENT DISCLOSURE

In an embodiment, the present disclosure provides a method and system for assisting a user in real-time for selecting products.

In an embodiment, the present disclosure considers health condition and a health goal associated with the user while assisting the user in selecting products and hence aides the user in making a quick decision in selecting a product as the user need not review information of the product in detail.

In an embodiment, the present disclosure reduces time taken by the user in selecting a product.

In an embodiment, the present disclosure enable retailers to engage the users by providing a personalized application for selecting products based on a health goal and a health condition associated with the user.

The terms “an embodiment,” “embodiment,” “embodiments,” “the embodiment,” “the embodiments,” “one or more embodiments,” “some embodiments,” and “one embodiment” mean “one or more (but not all) embodiments of the present disclosure” unless expressly specified otherwise.

The terms “including,” “comprising,” “having,” and variations thereof mean “including but not limited to,” unless expressly specified otherwise. The enumerated listing of items does not imply that any or all the items are mutually exclusive, unless expressly specified otherwise.

The terms “a,” “an,” and “the” mean “one or more,” unless expressly specified otherwise.

A description of an embodiment with several components in communication with each other does not imply that all such components are required. On the contrary, a variety of optional components are described to illustrate the wide variety of possible embodiments of the present disclosure.

When a single device or article is described herein, it will be clear that more than one device/article (whether they cooperate) may be used in place of a single device/article. Similarly, where more than one device or article is described herein (whether they cooperate), it will be clear that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the present disclosure need not include the device itself.

Finally, the language used in the specification has been principally selected for readability and instructional purposes, and it may not have been selected to delineate or circumscribe the inventive subject matter. It is therefore intended that the scope of the present disclosure be limited not by this detailed description, but rather by any claims that issue on an application based here on. Accordingly, the embodiments of the present disclosure are intended to be illustrative, but not limiting, of the scope of the present disclosure, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of assisting a user in real-time for selecting products, the method comprising:
receiving, by an assisting system in a device associated with a user, information relating to a set of nutrients associated with a selected product selected by the user;
causing, by the assisting system, the device to display, using an augmented reality (AR) technique, the information and at least one of a name or an image, the at least one of the name or the image associated with the selected product;
retrieving, by the assisting system, health data associated with the user, the health data comprising at least one of: one or more health conditions associated with the user, one or more medical conditions associated with the user, one or more medications associated with the user, or a health goal associated with the user, the health data retrieved from a user profile database associated with the assisting system;
identifying, by the assisting system, pre-stored nutrients relating to the set of nutrients;
analyzing, by the assisting system, the information based on a health effect of the pre-stored nutrients;
identifying, by the assisting system, one of an expected first impact on the health data or an expected second impact on the health data; and
determining, by the assisting system, that the selected product is suitable for the user upon identifying the expected first impact,
wherein the expected second impact is different from the expected first impact.

2. The method of claim 1, wherein:
the expected first impact is a positive impact expected to improve the health of the user; and
the expected second impact is a negative impact expected to worsen the health of the user.

3. The method of claim 2, further comprising determining, by the assisting system, that the selected product is unsuitable for the user upon identifying the expected second impact.

4. The method of claim 2, further comprising causing, by the assisting system, the device to provide a notification using the AR technique to the user when the selected product is determined as being one of suitable or unsuitable.

5. The method of claim 2, wherein the information is received upon scanning the selected product using an image recorder of the device or a barcode scanner.

6. The method of claim 2, further comprising communicating, by the assisting system, with a product database comprising:
one or more general health conditions;
the pre-stored nutrients, the pre-stored nutrients comprising:
pre-stored nutrients expected to positively impact the one or more general health conditions;
pre-stored nutrients expected to negatively impact the one or more general health conditions;
pre-stored nutrients that are suitable for the one or more medications;
pre-stored nutrients that are unsuitable for the one or more medications; and
pre-stored nutrients affecting the one or more medical conditions and the health goal;
a level of each of the pre-stored nutrients expected to positively impact the one or more general health conditions;
one or more first products comprising the pre-stored nutrients;
a level of each of the pre-stored nutrients expected to negatively impact the one or more general health conditions; and
one or more second products comprising the pre-stored nutrients expected to negatively impact the one or more general health conditions.

7. The method of claim 6, further comprising:
comparing, by the assisting system, the one or more general health conditions to the one or more health conditions;
identifying, by the assisting system, one or more matching general health conditions from the one or more general health conditions that match with the one or more health conditions;
identifying, by the assisting system, the pre-stored nutrients;
identifying, by the assisting system, the level of the pre-stored nutrients that positively impact the one or more matching general health conditions; and
recommending, by the assisting system, the one or more first products.

8. The method of claim 2, wherein identifying the one of the positive impact on the health data or the negative impact on the health data comprises:
comparing, by the assisting system, the one or more health conditions with a product database to determine one or more matched general health conditions;
comparing, by the assisting system, the set of nutrients with the product database to determine one or more matched pre-stored nutrients;
identifying the expected first impact when the matched pre-stored nutrients are expected to positively impact the one or more matched general health conditions; and
identifying the expected second impact when the matched pre-stored nutrients are expected to negatively impact the one or more matched general health conditions.

9. An assisting system for assisting a user with selecting products in real time, the assisting system comprising:
a display capable of performing an augmented reality (AR) technique;
a processor; and
a memory communicatively coupled to the processor, the memory storing processor-executable instructions, which, on execution, cause the processor to:
determine a selected product selected by the user;
receive information of a set of nutrients associated with the selected product;
cause the display to display the information using AR technique;
retrieve, from a user profile database associated with the assisting system, health data associated with the user, the health data comprising at least one of:
one or more health conditions associated with the user;
one or more medical conditions associated with the user;
one or more medications associated with the user; or
a health goal associated with the user;
determine pre-stored nutrients relating to the set of nutrients;
analyze the information based on a health effect of the pre-stored nutrients;
identify, based on the analysis of the information, one of an expected first impact of the set of nutrients on the health data or an expected second impact of the set of nutrients on the health data; and
detect the selected product as suitable for the user upon identifying the expected first impact,
wherein the expected second impact is different from the expected first impact.

10. The assisting system of claim 9, wherein:
the expected first impact is a positive impact expected to cause the health of the user to improve; and
the expected second impact is a negative impact expected to cause the health of the user to worsen.

11. The assisting system of claim 10, wherein the processor is further configured to detect the selected product as unsuitable for the user upon identifying the expected second impact.

12. The assisting system of claim 11, wherein the processor is further configured to cause the display to provide a notification to the user using the AR technique in response to detecting the selected product as suitable or unsuitable.

13. The assisting system of claim 10, further comprising a scanner configured to scan the selected product;
wherein the processor is further configured to receive the information upon scanning of the selected product.

14. The assisting system of claim 10, wherein the processor is communicable with a product database comprising:
one or more general health conditions;
the pre-stored nutrients, the pre-stored nutrients comprising:
the pre-stored nutrients, the pre-stored nutrients comprising:
pre-stored nutrients expected to positively impact the one or more general health conditions;
pre-stored nutrients expected to negatively impact the one or more general health conditions;
pre-stored nutrients that are suitable for the one or more medications;
pre-stored nutrients that are unsuitable for the one or more medications; and
pre-stored nutrients affecting the one or more medical conditions and the health goal;
a level of each of the pre-stored nutrients expected to positively impact the one or more general health conditions;
one or more first products comprising the pre-stored nutrients;
a level of each of the pre-stored nutrients expected to negatively impact the one or more general health conditions; and
one or more second products comprising the pre-stored nutrients expected to negatively impact the one or more general health conditions.

15. The assisting system of claim 14, wherein the processor is further configured to:
compare the one or more general health conditions to the one or more health conditions;
identify one or more matching general health conditions from the one or more general health conditions that match with the one or more health conditions;
identify the pre-stored nutrients;
identify the level of the pre-stored nutrients that positively impact the one or more matching general health conditions; and
recommend the one or more first products.

16. The assisting system of claim 10, wherein the processor identifies the one of the expected first impact on the health data or the expected second impact on the health data by:
comparing the one or more health conditions with a product database to determine one or more matched general health conditions;
comparing the set of nutrients with the product database to determine one or more matched pre-stored nutrients;
identifying the expected first impact when the matched pre-stored nutrients expected to positively impact the one or more matched general health conditions; and
identifying the expected second impact when the matched pre-stored nutrients expected to negatively impact the one or more matched general health conditions.

17. A non-transitory computer readable medium including instructions stored thereon that when executed by at least one processor causes an assisting system for assisting a user in real-time for selecting products to:
receive information of a set of nutrients associated with a selected product selected by the user;
display, using an augmented reality (AR) technique, the information and at least one of a name of the selected product or an image of the selected product;
retrieve, from a user profile database associated with the assisting system, health data associated with the user, the health data comprising at least one of:
one or more health conditions associated with the user;
one or more medical conditions associated with the user;
one or more medications associated with the user; or
a health goal associated with the user;
analyze the information based on a health effect of pre-stored nutrients relating to the set of nutrients;
identify, based on the analysis, one of an expected first impact of the set of nutrients on the health data or an expected second impact of the set of nutrients on the health data; and
indicate the selected product as suitable for the user upon identifying the expected first impact;
wherein the expected second impact is different from the expected first impact.

18. The non-transitory computer readable medium of claim 17, wherein:
the expected first impact is a positive impact expected to improve the health of the user; and
the expected second impact is a negative impact expected to worsen the health of the user.
